(19) United States
(12) Patent Application Publication
(10) Pub. No.: US 2012/0197157 A1
(43) Pub. Date: Aug. 2, 2012

(54) Distal Tip Configurations for Biopsy with EUS FNA

(76) Inventors: Shawn Ryan, Upton, MA (US); Caroline Gaudet, West Roxbury, MA (US); Kayla Burnim, Somerville, MA (US); Andrew Whitney, Douglas, MA (US); Barry Weitzner, Acton, MA (US)

(21) Appl. No.: 13/362,278

(22) Filed: Jan. 31, 2012

Related U.S. Application Data
(60) Provisional application No. 61/437,997, filed on Jan. 31, 2011.

(51) Int. Cl. A61B 10/02

(52) U.S. Cl. 600/567

(57) ABSTRACT

A biopsy device includes a needle extending longitudinally from a proximal end to a distal end and including a channel extending therethrough and a stylet extending through the channel of the needle and including a coil at a distal end thereof such that rotation of the stylet relative to the needle moves the coil from a closed configuration in the channel to a tissue collecting configuration in which the coil extends distally past the distal end of the needle to be inserted into a target tissue.

### PRIORITY CLAIM

The present application claims the priority to the U.S. Provisional Application Ser. No. 61/437,997, entitled “Distal Tip Configurations For Biopsy With EUS FNA” filed Jan. 31, 2011. The specification of the above-identified application is incorporated herewith by reference.

### BACKGROUND

Needle biopsies are common for the diagnosis and staging of disease. One type of biopsy is Endoscopic UltraSound Fine Needle Aspiration (EUS FNA), which involves the insertion of a needle under ultrasound guidance so that the physician may visualize the position of the needle in relation to target tissue and surrounding structures. EUS FNA procedures are used to ensure that the correct tissue is sampled and to minimize risk to the patient. Although EUS FNA is a highly sensitive and specific procedure, it is still difficult to acquire a sample in certain clinical situations. For example, different tip geometries may be suited for different situations due to tissue density and fibrosis. In addition, samples are most typically cytological, which may be difficult to handle.

### SUMMARY OF THE INVENTION

The present invention is directed to a biopsy device, comprising a needle extending longitudinally from a proximal end to a distal end and including a channel extending therethrough and a stylet extending through the channel of the needle and including a coil at a distal end thereof such that rotation of the stylet relative to the needle moves the coil from a closed configuration in the channel to a tissue collecting configuration in which the coil extends distally past the distal end of the needle to be inserted into a target tissue.

The present invention is further directed to a biopsy stylet sized and shaped for slidable insertion through a channel of a biopsy needle, the stylet including a coil at a distal end thereof such that rotation of the stylet relative to the needle moves the coil from a closed configuration in the channel to a tissue collecting configuration in which the coil extends distally past a distal end of the needle to be inserted into a target tissue

### BRIEF DESCRIPTION OF THE DRAWINGS

FIG. 1 shows a perspective view of a device according to a first exemplary embodiment of the present invention;

FIG. 2 shows a perspective view of a stylet of the device of FIG. 1;

FIG. 3 shows a cross-sectional perspective view of an actuating mechanism according to an exemplary embodiment of the present invention;

FIG. 4 shows a perspective view of an actuating mechanism according to an alternate embodiment of the present invention;

FIG. 5 shows a perspective view of a device according to a second exemplary embodiment of the present invention;

FIG. 6 shows a first longitudinal side plan view of the device of FIG. 5;

FIG. 7 shows a second longitudinal side plan view of the device of FIG. 5, the device rotated approximately 90 degrees relative to the first longitudinal side plan view;

FIG. 8 shows a perspective view of a device according to a third exemplary embodiment of the present invention;

FIG. 9 shows a perspective view of a device according to a fourth exemplary embodiment of the present invention; and

FIG. 10 shows a perspective view of a device according to a fifth exemplary embodiment of the present invention.

### DETAILED DESCRIPTION

The present invention may be further understood with reference to the following description and the appended drawings, wherein like elements are referred to with the same reference numerals. The present invention relates to devices for needle biopsies and, in particular, relates to a biopsy device including improved distal tip features for tissue acquisition. Exemplary embodiments of the present invention describe distal tip geometries of stylets used in conjunction with an endoscopic needle device. It should be noted that the terms “proximal” and “distal,” as used herein, are intended to refer to a direct toward (proximal) and away from (distal) a user of the device.

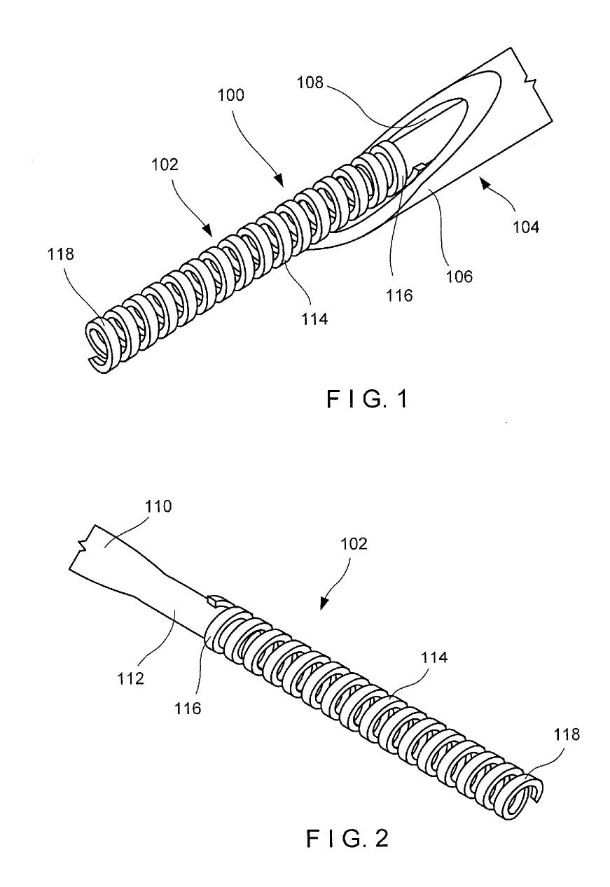

As shown in FIGS. 1-2, a device 100 according to a first exemplary embodiment comprises a stylet 102 configured for use in conjunction with a needle 104 such as, for example, an EUS FNA needle. The needle 104 extends longitudinally from a proximal end (not shown) to a distal end 106 and includes a channel 108 extending therethrough. The needle 104 may be inserted into a target area of a body to gain and maintain access to a target area in which tissue to be sampled is located. The stylet 102 is inserted through the channel 108 to sample tissue from the target area. The needle 104 is axially flexible along its length thereof so that it may be inserted through the body to the target area along a tortuous path (e.g., through a natural body lumen). The needle 104 may be a formed of, for example, a polymer, stainless steel, a chromium cobalt alloy, nitinol or any combination of materials and may be constructed in the same manner as currently available EUS FNA needles. The distal end 106 may be cut at an angle to facilitate penetration of the distal end 106 into tissue. In another embodiment, the distal end 106 may be tapered. A surface surrounding the opening in the distal end 106 is preferably formed as a cutting edge to aid in severing from surrounding tissue, tissue grasped by the stylet 102 as will be described in more detail below. As would be understood by those skilled in the art, the stylet 102 and the needle 104 may include handles at proximal ends thereof, which are connectable to one another to facilitate coupling of the stylet 102 and the needle 104.

The stylet 102, as shown in FIG. 2, includes a flexible shaft 110 formed as, for example, a wire extending longitudinally from a proximal end (not shown) to a distal end 112. A proximal end 116 of the coil 114 is coupled to the distal end 112 of the shaft 110 and the coil 114 extends from the shaft 110 along a substantially helical path from the coil proximal end 116 to a coil distal end 118. A pitch of the coil 114 may be altered along a length thereof from the proximal end 116 to the distal end 118 to vary a flexibility of the coil 114 along a length thereof and/or affect the an ability of the coil 114 to puncture tissue. In another embodiment, the coil 114 may be tapered from the proximal end 116 to the distal end 118 to facilitate puncturing of tissue. In addition, edges of the coil 114 may be sharpened. In an alternate embodiment, a leading edge at the distal end 118 may be sharpened. The distal end 112 of the shaft 110 may be tapered such that, when the coil 114 is received around the distal end 112, an inner diameter of the coil 114 is substantially equal to an outer diameter of the proximal portion of the shaft 112. Alternatively, the coil 114 may be integrally formed with the distal end 112 of the shaft 110. For example, the coil 114 may be integrally formed with the shaft 110 by tapering the distal end 112 (e.g., swaging, drawing, machining) and then coiling and heat setting the tapered portion. In another example, a core may be drilled out of the distal end 112 of the shaft 110 and a spiral configuration cut (e.g., laser, EDM, milled) from the cored distal end 112. Additionally, the coils 114 may be spaced apart from one another such that tissue may be sampled between each of the coils 114.

The stylet 102 is slidably received in the channel 108 of the needle 104 and is movable between a closed configuration for inserting the device 100 into the body and withdrawing the device 100 therefrom and a tissue sampling configuration for sampling tissue. In the closed configuration, the coil 114 is withdrawn into the channel 108 with the coil distal end 118 located within the opening in the distal end 106 of the needle 104 to prevent non-targeted tissue from entering the channel 108 as the needle is advanced to the target area through non-targeted tissue. Once the distal end 106 of the needle 104 has reached the target area, the stylet 102 is advanced distally to move the coil 114 into the target tissue. For example, the shaft 110 may be screwed into the target tissue by rotating the shaft 110. That is, the stylet 102 may be rotated relative to the needle 104 about a longitudinal axis thereof as the stylet 102 is moved distally relative to the needle 104 to facilitate penetration of the target tissue via the coil 114. The stylet 102 may be advanced and/or rotated to penetrate the target tissue.

Once the coil 114 has penetrated the target tissue, the stylet 102 may be retracted proximally into the closed configuration via a proximal motion of the shaft 110 drawing target tissue grasped by the coil 114 into the channel 108. Once target tissue has been grasped by the coil 114, the stylet 102 may be moved proximally manually or via an actuating mechanism 120. As the coil 114 is drawn into the needle 104, a cutting edge on the distal end 106 of the needle 104 severs the tissue grasped by the coil from the surrounding tissue such that the cut portion of tissue is housed within the needle 104 until the device 100 is removed from the body. In another embodiment, the needle 104 may be moved distally relative to the stylet 102, moving the needle 104 over the coil 114 and the tissue grasped thereby to capture a tissue sample within the needle 104 and sever the captured tissue from surrounding tissue.

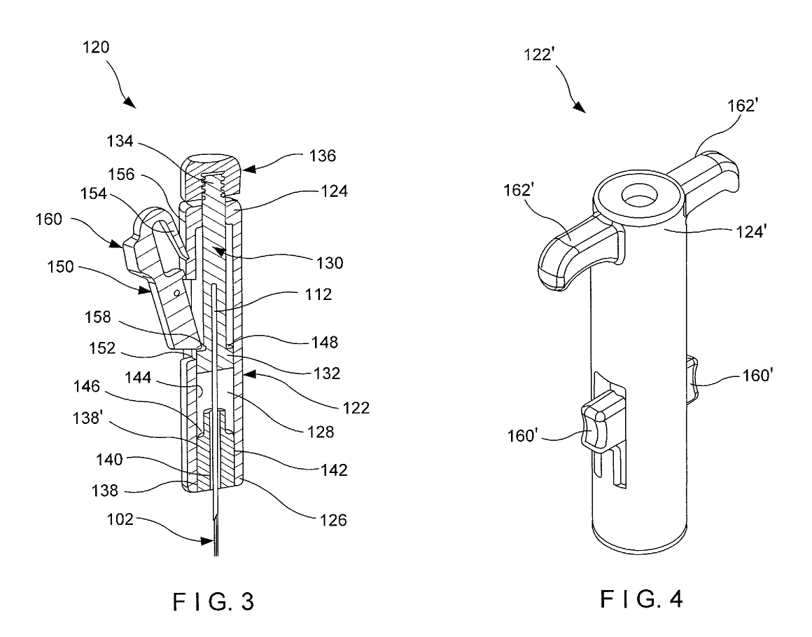

The actuating mechanism 120 may be an add-on feature or a separate device coupled to a proximal end of the device 100 to draw the stylet 102 proximally relative to the needle 104, reverting the device 100 to the closed configuration. For example, as shown in FIG. 3, the actuating mechanism 120 may be attached to the proximal end of the stylet 102 with the stylet 102 inserted through the channel 108 of the needle 104. The actuating mechanism 120 comprises a housing 122, a piston 130 and a lever 150. The housing 122 extends from a proximal end 124 to a distal end 126 with a channel 128 extending therethrough. The piston 130 extends from a proximal end 134 to a distal end 132 with the distal end 132 movably housed within the channel 128 while a proximal end 134 extends proximally past the proximal end 124 of the housing 122 to engage a cap 136. The distal end 132 includes a shoulder 148 extending radially outward such that the distal end 132 of the piston is larger than a remaining portion of the piston 130 to engage an inner surface of the channel 128. The proximal end of the stylet 102 is fixed to the piston 130 such that moving the piston 130 longitudinally relative to the housing 122 moves the stylet 102 between the tissue sampling configuration and the closed configuration.

The actuating mechanism 120 further includes a female luer connection 138 coupling to a male luer 138′ of the needle device. The stylet 102 passes through a lumen 140 of the male luer 138′ into the channel 128. For example, the female luer connection 138 may include a threaded inner surface 144 engaging an outer surface 142 of the male luer 138′. It will be understood by those of skill in the art, however, that the luer 138 may engage the housing 122 in any number of ways. A spring (not shown) may be housed within the channel 128, between a proximal end 146 of the male luer 138′ and the distal end 132 of the piston 130, biasing the piston 130, and thus the stylet 102, toward the closed configuration. The spring may be, for example, a helical, coil spring biasing the piston 130. In an alternative embodiment, the spring may include a gas that may be captured in, for example, a gas piston. It will be understood by those of skill in the art that the spring may be any biasing element that biases the piston 130, and thereby the stylet 102, toward the closed configuration.

The lever 150 is pivotably attached to a side of the housing 122 and extends through an opening 152 in a side of the housing 122. The lever 150 includes a biasing element 154 contacting an outer surface 156 of the housing 122 to bias the lever 150 toward a position angled relative to a longitudinal axis of the housing 122 so that an engaging element 158 of the lever extends through the opening 152 and engages a side of the piston 130. When the actuating mechanism 120 is in the closed configuration, the piston 130 is moved proximally through the channel 128 with the engaging element 158 of the lever 150 engaging the radially protruding distal end 132 of the piston. In this position, the lever 150 is rotated to a position substantially parallel to the longitudinal axis of the housing 122 (i.e., substantially parallel to the needle 102 extending therethrough). To move the device to the tissue sampling configuration, a user pushes the cap 136 distally toward the proximal end 124 of the housing 122 moving piston 130 distally through the housing 122 until the engaging element 158 of the lever 150 moves out of contact with the radially protruding end 132 of the piston 130. At this point, the biasing element 154 rotates the lever 150 (counterclockwise as seen in FIG. 3) so that the engaging element 158 moves radially inward to engage the reduced diameter portion of the piston 130 seated proximally of the shoulder 148. The spring (not shown) in the channel 128 urges the piston 130 against the engaging element 158, locking the device in the tissue sampling configuration. To withdraw the stylet 102 proximally into the closed configuration, the user simply rotates the lever 150 (clockwise as seen in FIG. 7) to disengage the engaging element 158 from the shoulder 148 so that the spring pushes the piston 130 proximally and the stylet 102 is drawn proximally into the needle 104 (i.e., into the closed configuration).

As described above, in the tissue sampling configuration, the distal end 118 of the coil 114 extends distally past the distal end 106 of the needle 104 to penetrate the target tissue. Once the target tissue is penetrated by the coil 114 and thereby grasped by the coil 114, a user may press a button 158 on the lever 150 to release the engaging element 156 from the shoulder 148 and allow the piston 130 to move proximally into the closed configuration. This draws the stylet 102 along with the grasped target tissue proximally into the channel 108 of the needle 104. If additional tissue samples are desired, it will be understood by those of skill in the art that the stylet 102 may be removed from the body while the needle 104 remains therein. Thus, the needle 104 maintains the path to the target area within the body such that the stylet 102 may be repeatedly inserted therethrough to collect tissue samples.

As shown in FIG. 4, a housing 122′ of a device according to a further embodiment is substantially similar to the housing 122 of the actuating mechanism 120, but further includes ergonomic features to facilitate manipulation by a user. For example, the housing 122′ includes finger grips 160′ at a proximal end 124′ making the housing 122′ more easily gripped as the cap 136 is pressed into the tissue sampling configuration. As opposed to the single button of the mechanism 120, the housing 122′ may include dual buttons 160′ positioned on opposing sides of the housing 122′ facilitating the such that the dual buttons 160′ may be easily pressed as the housing 122′ is gripped by the user. It will be understood by those of skill in the art that the dual buttons 160′ will work in substantially the same manner as described above for the button 160 of the lever 150.

Although the device 100 has been described as used with the actuating mechanism 120, it will be understood by those of skill in the art that the device 100 may be moved between the closed and tissue sampling configurations using any actuating mechanism capable of drawing the stylet 102 into the channel 108 of the needle 104.

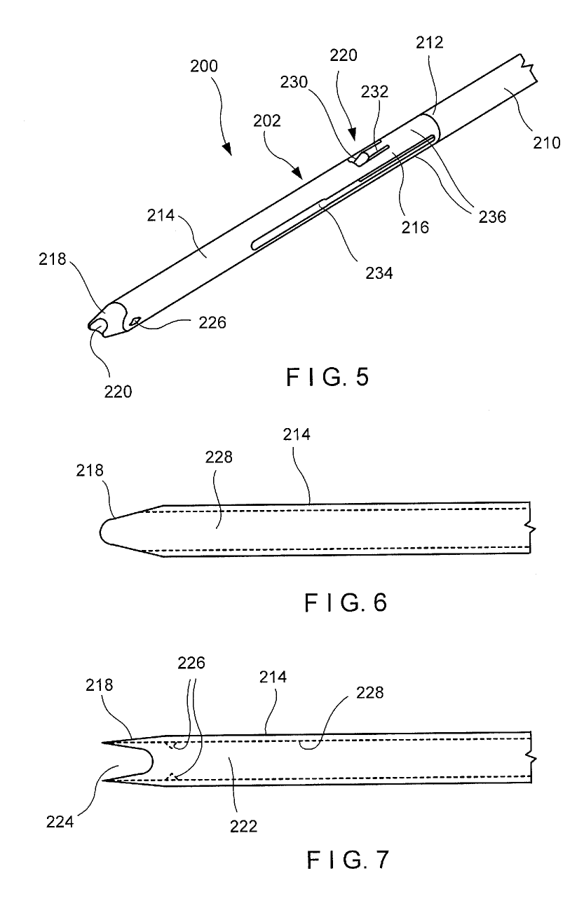

As shown in FIGS. 5-7, a device 200 according to another exemplary embodiment comprises a tube 202 that may be used with the needle 104, as described above in regard to the device 100. The tube 202 comprises a distal portion 214 releasably coupled to a distal end 212 of a proximal portion 210 via a coupling mechanism 220. Similarly to the device 100, the tube 202 may be longitudinally movable within the channel 108 of the needle 104 between a closed configuration and a tissue sampling configuration. Alternatively, the tube 202 may be used alone, without the needle 104, to sample tissue from the body.

The distal portion 214 extends from proximal end 216 to a distal tip 218 and includes a lumen 222 extending therethrough. The distal portion 214 may be formed as, for example, a hypotube, with a sharpened tissue penetrating distal tip 218. The distal tip 218 may be tapered and formed as a fish-mouth to facilitate insertion into a target tissue. The fish-mouth may be formed via an oscillating bevel as would be understood by those skilled in the art. For example, the distal tip 128 may include two opposing U-shaped slots 224 extending along a portion of a length thereof such that the distal tip 218 appears as a convex parabola in a first longitudinal side view, as shown in FIG. 6, and a concave parabola in a second longitudinal side view in which the tube 202 is rotated approximately 90 degrees about a longitudinal axis thereof, as shown in FIG. 7. The fish-mouth tip 218 and the lumen 222 permits tissue to be collected therewithin via a coring action. In particular, the tapering of the fish-mouth about an entire circumference thereof creates a sharp edge facilitating a clean cut at an edge of the core sample. In addition, cutting edges of the fish-mouth are oriented at different angles such that a cleaner cut is achieved when the tube 202 is rotated while being distally advanced. Slightly proximal of the distal tip 218, the distal portion 214 includes a protrusion 226 extending radially inward from an inner surface 228 of the lumen 222 configured to aid in tissue retention. The protrusion 226 may be formed as, for example, v-shaped cut along a wall of the distal portion 214 extending radially inward, as shown in FIG. 5. In another embodiment, as shown in FIG. 7, the protrusion 226 may be formed as a barb, spike, tooth or hook extending radially inward from the inner surface 228. It will be understood by those of skill in the art that the protrusion 226 may be any of a variety of shapes and configurations so long as tissue received within the lumen 222 at the distal tip 218 is retained therewithin via the protrusion 226. The distal portion 214 may include more than one protrusion 226 to aid in tissue retention. For example, the distal portion may include a pair of protrusions 226 diametrically opposed from one another within the lumen 222. The distal portion 214 may also include a pair of longitudinal slots 234 extending distally from the proximal end 216 and arranged on opposite sides of the distal portion 214 to facilitate attachment of the distal portion 214 to the proximal portion 210. The longitudinal slots 234 form jaws 236 at the proximal end 216 of the distal portion 214, which may be moved apart from one another to receive and accommodate the distal end 212 of the proximal portion 210 therebetween. It will be understood by those of skill in the art that at least the proximal end 216 of the of the distal portion 214 is formed of an elastic material, which permits the jaws 236 to revert to its initial position to hold the proximal portion 210 therebetween. A length of the slots 234 may be selected to facilitate this movement of the jaws 236.

The coupling mechanism 220 may be, for example, a latch mechanism including an opening 230 extending laterally through the proximal end 216 of the distal portion 214 sized and shaped to receive a tab 232 extending laterally outward from the distal end 212 of the proximal portion 210 to engage the opening 230. Thus, to couple the distal portion 214 to the proximal portion 210, the distal end 212 of the proximal portion 210 may be inserted into the proximal end 216 of the distal portion 214 until the tab 232 is engaged with the opening 230. To release the coupling mechanism 220, a user may press the tab 232 inward so that the tab 232 is disengaged from the opening 230 and the distal portion 214 is released from the proximal portion 210. It will be understood by those of skill in the art, however, that the coupling mechanism 220 any mechanism that facilitates releasable coupling between the distal portion 214 and the proximal portion 210. For example, in another embodiment, the coupling mechanism 220 may provide an annular snap fit or a threaded engagement. The distal portion 214 may be disengaged from proximal portion 210 aid in removal of the tissue sample from within the lumen 222 once the device 200 has been removed from the body. Upon disengagement from the proximal portion 210, the user is able to access the tissue collected within the lumen 222 from both the proximal and distal ends 216, 218 thereof.

Similarly to the device 100, in the closed configuration, the distal portion 214 of the tube 202 is housed substantially within the channel 108 of the needle 104 with the distal tip 218 residing in an opening in the distal end 106 of the needle 104. It will also be understood by those of skill in the art that the device 200 may be initially inserted into the body with a stylet including for example, a ball or conical tip, received within the lumen 222 thereof to prevent any tissue from entering the lumen 222 as the device 200 is being inserted to the target area. Once the device 200 has been positioned in the body, as desired, the device 200 is moved to the tissue sampling configuration by moving the tube 202 distally relative to the needle 104 so that the distal portion 214 extends into and penetrates the target tissue. As the distal portion 214 penetrates the target tissue, the target tissue is received within the lumen 222 and is retained therewithin via the protrusion 226. The tube 202 may also be rotated about a longitudinal axis thereof to aid in tissue removal. Once tissue has been collected within the distal portion 214, the tube 202 may be moved proximally relative to the needle 104 to revert back to the closed configuration. As the device 200 is moved back to the closed configuration, a cutting edge on the distal end 106 may sever the tissue received within the lumen 222 from the surrounding tissue. As discussed with respect to the device 100, the tube 202 may be moved proximally via a manual motion of the tube 202 or an actuating mechanism 120, 120′. Alternatively, the needle 104 may be moved distally over the tube 202 to cover the distal portion 214.

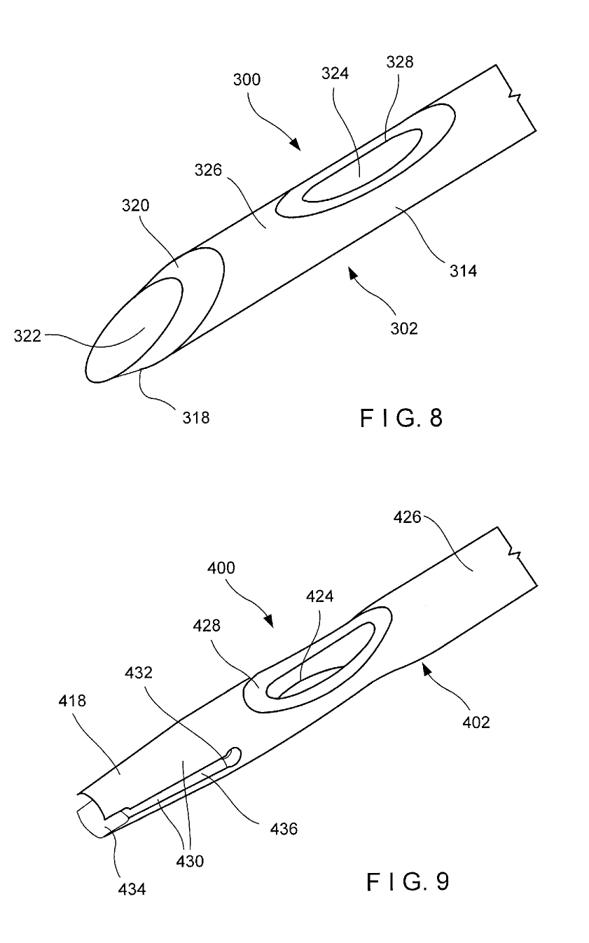

As shown in FIG. 8, a device 300 according to another exemplary embodiment of the present invention comprises a tube 302 that may be used in conjunction with the needle 104, as described above in regard to the device 100. Alternatively, the tube 302 may be used without the needle 104. The tube 302 extends longitudinally from a proximal end (not shown) to a distal tip 318 and includes a lumen 322 extending along at least a portion thereof from an opening at the distal tip 318. The tube 302 may be formed as, for example, a hypotube. The distal tip 318 may be cut at an angle of approximately 60 degrees relative to a longitudinal axis thereof and includes a bevel 320 extending therearound.

Slightly proximally of the distal tip 318, along a distal portion 314 thereof, the tube 302 includes a side opening 324 extending through a wall 326 thereof for receiving tissue into the lumen 322. The side opening 324 may include a cutting edge 328 for severing from surrounding tissue the tissue received through the side cut 324. In one exemplary embodiment, the side opening 324 may be substantially rounded, in the shape of, for example, a cylinder intersecting the wall 326 substantially perpendicular to a longitudinal axis of the tube 302 so that the cutting edge 328 of the side opening 324 extends about an entire perimeter thereof. Thus, a tissue sample may be cut via a variety of motions such as, for example, a forward and/or backward motion along with a rotational motion. However, it will be understood by those of skill in the art that the side opening 324 may be any of a variety of shapes so long as tissue may be received therethrough. In a preferred embodiment, the tube 302 includes two side openings 324, a second side opening 324 positioned along the tube 302 in a position substantially diametrically opposing the first side opening 324. It will be understood by those of skill in the art, however, that the tube 302 may include any number of side openings 324 in any of a variety of positions along the stylet.

In an alternate embodiment, the distal portion 314 of the tube 302 may be releasably coupled to a remaining portion of the tube 302, similarly to the tube 202. A releasable coupling aids in tissue removal from the lumen 322 after tissue collection. The tube 302 may be utilized in a manner substantially similar to the device 200. Similarly to the device 200, the device 300 is movable from a closed configuration to a tissue sampling configuration in which the distal tip 318 extends distally past the distal end 106 of the needle 104 to be inserted into a target tissue. The tube 302 may be moved distally and/or rotated relative to the needle 104 to collect tissue within the lumen 322 via the distal tip 318 and/or the side opening 324. Upon collection of the target tissue, the device 300 may be moved to the closed configuration and removed from the body, as described with respect to the device 100 above.

As shown in FIG. 9, a device 400 according to another exemplary embodiment of the present invention comprises a stylet 402 that may be used in conjunction with the needle 104, as described above with respect to the device 100, to move the device between a closed configuration and a tissue sampling configuration. Alternatively, the stylet 402 may be used without the needle 104. The stylet 402 may be substantially similar to the tube 302 extending longitudinally from a proximal end (not shown) to a distal end 418 and including a lumen 422 extending through at least a distal portion thereof proximally from an opening at the distal end 418. Similarly to the tube 302, the stylet 402 includes a side opening 424 in a wall 426 thereof through which tissue is received into the lumen 422. The side opening 424 may be slightly proximal of the distal tip 418 and include a cutting edge 428 providing additional cutting surfaces suitable for utilization during insertion, rotation and/or removal. The side opening 424, however, may be in the shape of a cylinder intersecting the wall 426 at an angle of approximately 45 degrees relative to a longitudinal axis of the stylet 402. Also similarly to the tube 302, the tube 402 may preferably include a second side opening diametrically opposed from the first side opening 424. It will be understood by those of skill in the art, however, that the tube 402 may include any number of side openings 424 arranged in any of a variety of positions therealong.

The distal end 418 of the tube 402 may be tapered and includes a plurality of tips 430 formed via at least one longitudinal slot 432 extending therethrough. For example, the longitudinal slot 432 may cut the distal end 418 in half, forming two tips 430 which are biased in a position in which the tips 430 that are separated, but set close to one another. Each of the tips 430 may have a leading edge 434 along an inner surface 436 thereof such that insertion of the distal end 418 into a target tissue causes the tips 430 to move away from one another into an open configuration to receive tissue therebetween. Removal of the tube 402 from the target tissue by, for example, moving the device 400 into the closed configuration in which the distal end 418 of the tube 402 is received within the needle 104, will move the tips 430 back to the biased configuration, gripping tissue therebetween. Thus, the tube 402 may collect tissue via both the tips 430 and the side opening 424.

The tube 402 may be used with the needle 104 in a manner substantially similar to the devices 200 and 300, as described above. Specifically, the tube 402 may be moved distally out of the needle 104 to be inserted into the target tissue. The insertion and/or rotation of the tube 402 facilitates collection of tissue within the lumen 422 via the side opening 424 and the tips 430. Once tissue has been collected, as desired, the device 400 may be moved to the closed configuration, as described above with respect to the device 100, while also severing the collected tissue from the surrounding tissue.

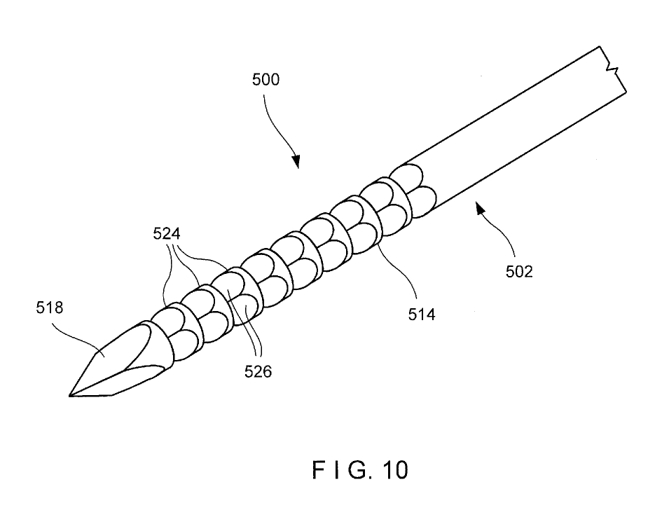

As shown in FIG. 10, a device 500 according to another exemplary embodiment of the present invention comprises a stylet 502 that may be utilized in conjunction with a needles, such as, for example, the needle 104, as described above in regard to the device 100. The stylet 502 extends longitudinally from a proximal end (not shown) to a distal end 514 and includes a plurality of ribs 524 extending along a portion of a length of the distal end 514. The distal end 514 may also include a trocar point 518 so that, in a preferred embodiment, the stylet 502 would be used in conjunction with a needle including a corresponding Franseen grind. The device 500 may be moved between a closed configuration, in which the distal end of the stylet 502 closes the distal opening of the needle 104 to prevent unwanted tissue from entering the channel 108, and a tissue sampling configuration, in which the distal end 514 extends distally past the distal end 106 of the needle 104.

Each of the ribs 524 extends about a periphery of the stylet 502 and may be formed of a plurality of planar surfaces 526 for improved visibility. In a preferred embodiment, the ribs 524 may be four-sided facets such that each of the ribs 524 includes four planar surfaces 524. The ribs 524 may be back-cut such that the distal end 514 is easily inserted into tissue when moved distally into the tissue sampling configuration while cutting and retaining tissue when moved proximally into the needle 104, into the closed configuration. As the stylet 502 is moved proximally, the tissue is collected between each of the ribs 524.

The device 500 may be utilized in a manner substantially similar to the device 100. In particular, the device 500 is inserted into the body int the closed configuration, until a distal end thereof is adjacent a target area. The device 500 may then be moved to the tissue sampling configuration such that the distal end 514 of the stylet pierces tissue and is inserted therein. The stylet 502 may be reverted to the closed configuration using any of the methods, as described above in regard to the device 100. Moving the device 500 into the closed configuration captures the tissue sample within the needle 104.

It will be understood by those of skill in the art that individual features of the embodiments described above may be omitted and or combined to form alternate embodiments. For example, an alternate embodiment may include a device comprising hypotube including a plurality of tips at a distal end thereof, as described in regard to the tube 402, and which is releasably couplable to a proximal portion thereof, as described in regard to the tube 202. It will be understood by those of skill in the art that there exists a variety of possible combinations of features.

It will be understood by those skilled in the art that various modification can be made in the structure and the methodology of the present invention, without departing from the spirit or scope of the invention. Thus, it is intended that the present invention cover the modifications and variations of this invention provided that they come within the scope of the appended claims and their equivalents.

What is claimed is:

1. A stylet for insertion into a biopsy needle, comprising:
a coil at a distal end thereof such that advancement of the stylet relative to the needle moves the coil from a closed configuration in the needle to a tissue collecting configuration in which the coil extends distally past the distal end of the needle.

2. The stylet of claim 1, wherein the coil is rotatable relative to the needle to be inserted into a target tissue.

3. The stylet of claim 1, further comprising:
a shaft such that the coil is attachable to a distal end of a shaft.

4. The stylet of claim 1, wherein the coil extends substantially helically about a longitudinal axis of the stylet.

5. A biopsy device, comprising:
a tissue collecting element including a distal portion extending longitudinally from a proximal end to a distal end and including a lumen extending therethrough, the distal end including a slot extending along a portion of a length thereof to form opposing jaws for collecting a target tissue therebetween and into the lumen.

6. The device of claim 5, wherein the slot is substantially U-shaped such that a distal face of the tissue collecting element oscillates about a longitudinal axis thereof.

7. The device of claim 5, wherein the tissue collecting element includes a first protrusion extending radially inward from an inner surface of the lumen, the first protrusion configured to grasp tissue received within the lumen.

8. The device of claim 7, wherein tissue collecting element includes a second protrusion extending radially inward from the inner surface of the lumen, the second protrusion being substantially diametrically opposed from the first protrusion.

9. The device of claim 7, wherein first protrusion is configured as one v-shaped cut along a wall of the tissue collecting element, a barb, spike, hook and tooth.

10. The device of claim 5, wherein the distal portion is releasably couplable to a proximal portion of the tissue collecting element.

11. The device of claim 10, wherein the distal portion is couplable portion via one of a latch mechanism, a snap-fit and a thread.

12. The device of claim 5, wherein the distal portion includes a first side opening extending through a wall thereof, the first side opening including a cutting edge extending about a periphery thereof.

13. The device of claim 12, wherein the first side opening is in the shape of a cylinder intersecting the wall substantially perpendicular to a longitudinal axis of the tissue collecting element.

14. The device of claim 12, wherein the first side opening is in the shape of a cylinder intersecting the wall at an angle of approximately 45 degrees relative to a longitudinal axis of the tissue collecting element.

15. The device of claim 12, wherein the tissue collecting element includes a plurality of openings extending through a portion of the wall opposing the first side opening, the second side opening including a cutting edge extending about a periphery thereof.

16. The device of claim 5, wherein the opposing jaws include leading edges along an inner surface thereof such that moving the tissue collecting element distally through a target tissue separates the opposing jaws to receive the target tissue therebetween.

17. The device of claim 5, wherein the tissue collecting element is sized and shaped for insertion through a channel of a needle.

18. A stylet for insertion into a biopsy needle, comprising:
a tissue collecting element including a distal portion extending longitudinally from a proximal end to a distal end and including a lumen extending therethrough, the distal end including a slot extending along a portion of a length thereof to form opposing jaws for collecting a target tissue therebetween and into the lumen.

19. A biopsy stylet sized and shaped for slidable insertion through a channel of a biopsy needle, the stylet including a coil at a distal end thereof such that rotation of the stylet relative to the needle moves the coil from a closed configuration in the channel to a tissue collecting configuration in which the coil extends distally past a distal end of the needle to be inserted into a target tissue.

20. The stylet of claim 20, wherein the coil includes a varying pitch along a length thereof.

21. The stylet of claim 20, wherein the coil is tapered from a proximal end to a distal end thereof.

22. The stylet of claim 20, wherein the coil includes a sharp edge.

23. The stylet of claim 23, wherein a distal tip of the coil includes a sharp edge.
